(19) United States
(12) Patent Application Publication
(10) Pub. No.: US 2016/0060595 A1
(43) Pub. Date: Mar. 3, 2016

(54) METHODS AND MEDIA FOR DIFFERENTIATING EYE CELLS

(71) Applicant: TAMPEREEN YLIOPISTO, Tampereen yliopisto (FI)

(72) Inventors: Alexandra MIKHAILOVA, Tampere (FI); Tanja ILMARINEN, Turenki (FI); Heli SKOTTMAN, Lempaala (FI)

(21) Appl. No.: 14/782,104

(22) PCT Filed: Jan. 23, 2014
(86) PCT No.: PCT/FI2014/050053

§ 371 Date: Oct. 2, 2015

(30) Foreign Application Priority Data
Apr. 3, 2013 (FI) 20135318

(51) Int. Cl. C12N 5/079

(52) U.S. Cl. CPC C12N 5/0621; C12N 2501/115; C12N 2506/03; C12N 2501/11; C12N 2501/415; C12N 2501/40

(57) ABSTRACT

Here is provided a novel differentiation protocol, which was experimentally shown to give rise to corneal epithelial precursor cells or early pigmented RPE precursor cells in defined and xeno-free conditions. The early precursor cells may be further maturated towards corneal epithelium cells, stratified corneal epithelium or mature RPE cells. Such cells may contribute to treatment and research of corneal and retinal conditions, diseases, pathologies as well as toxicology and drug development.

### FIELD OF THE INVENTION

The technical field concerned is treatment and study of the eye, more specifically corneal and retinal diseases and disorders. The present description is related to differentiation of stem cells into eye precursor cells and further into differentiated eye cells, such as corneal epithelial cells or retinal pigment epithelial cells. Accordingly, here is provided means and methods contributing to fast and effective induction, maturation and differentiation.

### BACKGROUND OF THE INVENTION

The cornea is located at the front surface of the eye and is multi-layered, transparent and avascular in structure. The main functions of the cornea are to protect the eyeball and its contents, while allowing accurate focusing of light to produce a sharp image on the retina. The cornea consists of three cellular layers, namely epithelium, stroma and endothelium, separated by two acellular layers—Bowman's layer, and Descemet membrane. As the outermost layer, corneal epithelium is exposed to the external environment and thereby needs to be rapidly regenerating and stratified. Similarly to the epidermis, lens and conjunctival epithelium, corneal epithelium originates from the surface ectoderm. However, detailed developmental mechanisms and signaling routes remain unknown.

In a prior art publication, Hayashi et al, 2012 reported corneal cell differentiation on PA6 cells (feeder cells of mouse origin) taking 12-16 weeks. The differentiation efficiency measured with expression of CK12 protein was less than 15%. Better efficiency and xeno-free conditions are still desired.

Hanson et al, 2012 published differentiation on Bowman's membrane for 25 days. The efficiency was not, however, reported. The Bowman's membrane is known being unstable, which casts uncertainty to the procedure.

Yoshida et al, 2011 managed to produce precursor cells by differentiation of mouse induced pluripotent stem cells through cultivation on PA6 cells (feeder cells of mouse origin). Differentiation took about 60 days. There still is need for xeno-free differentiation conditions.

Ahmad et al, 2007 published culturing of hESC on collagen IV. They used medium conditioned by the limbal fibroblasts which resulted in the loss of pluripotency and differentiation into epithelial like cells. They reported differentiation efficiency of 50% on day 5 and 10% on day 21 as measured by expression of proteins CK3/12. Use of a medium which requires donated limbal cells can be considered problematic. Moreover, there is significant biological variation between batches of limbal cells.

Retinal pigment epithelium (RPE) is an epithelial cell monolayer located between the neural retina and choriocapillaris. RPE provides essential support for the long-term preservation of retinal integrity and visual functions by absorbing stray light, regenerating visual pigment, supplying nutrients, secreting growth factors, and phagocytosing the shed photoreceptor outer segments (POS). Dysfunctional RPE causes impairment and death of the photo-receptor cells, leading to deterioration or total loss of vision. These mechanisms play an important role in the pathogenesis of retinal diseases like age-related macular degeneration (AMD), which is the leading cause of blindness in the developed world.

Human pluripotent stem cells may serve as an unlimited source of RPE cells for transplantation. Several groups have reported successful RPE differentiation originating from human embryonic stem cells (hESCs) and human induced pluripotent stem cells (hiPSCs). However, current differentiation methods for RPE cells mainly rely either on spontaneous differentiation processes favoring neuroectodermal lineage, which is characteristic of hESCs, or on a complex mixture of several different growth factors, inhibitors or other functional ingredients.

Osakada et al., 2008 and 2009 succeeded in enhancing RPE differentiation efficiency with a prolonged culture period and cell culture supplements. Studied supplements include: nicotinamide (NIC), Activin A, transforming growth factor beta (TGFβ), Wnt signaling inhibitor casein kinase I inhibitor (CKI)-7, dickkopf-related protein-1 (Dkk-1), Lefty-A, fibroblast growth factor antagonist Y-27632, and nodal signaling inhibitor SB431542. Regardless of the improvement of the differentiation efficacy, reaching a sufficient amount of maturated cells with RPE characteristics still demanded long-term differentiation processes.

WO 2013/184809 discloses a relatively fast method for the derivation of RPE cells from pluripotent cells. However, the method is very complex with at least four different stages and four different culture media, each characterized by a unique combination of active supplement.

Xeno-products and undefined factors used in many differentiation processes pose further challenges, because animal-derived components may carry factors such as sialic acid or Neu5Gc, causing unwanted immunogenicity of the cells or even animal pathogens. Fetal bovine serum (FBS) is widely used, at least in some stages of the culture of RPE cells. KnockOut™ Serum Replacement (KO-SR), used to replace FBS in many laboratories, still contains BSA (BSA) and bovine transferrin. In addition, most of the current differentiation methods utilize a culture environment produced by mouse embryonic fibroblast (MEF) cells widely used as feeder cells for hESCs and hiPSCs.

WO 2010/144696 relates to methods and compositions for producing neural cells from stem cells, while WO 2012/103012 relates to generating inner ear cells. Among potential differentiation-inducing agents, both publications disclosed bFGF, a TGF-beta inhibitor, and a Wnt inhibitor.

There still is need for supplies and methods for culturing stem cells via controlled differentiation to produce cells contributing to treatment and research of retinal and corneal epithelium conditions, diseases, pathologies, as well as toxicology and drug development. Further, cell culture media for use in such methods are equally desired. Further, avoidance of undefined components, such as amniotic membrane or conditioned medium is also an important aim.

### BRIEF DESCRIPTION OF THE INVENTION

It is thus an object of the present invention to provide means and methods effective to differentiate stem cells into eye precursor cells and further into corneal epithelial or early pigmented RPE precursor cells, which may then be maturated into mature corneal epithelial cells and mature RPE cells, respectively.

Thus, in one aspect, the present invention provides a method of producing eye precursor cells. Said method comprises culturing pluripotent stem cells in an induction medium comprising TGF-beta inhibitor, a Wnt inhibitor and a fibroblast growth factor.

In another aspect, the present invention provides a method of producing differentiated eye cells. Said method comprises:

a) providing eye precursor cells by a method set forth above;

b) culturing the cells

i) for producing corneal epithelial precursor cells, in a cell culture medium comprising one or more supplements selected from the group consisting of epidermal growth factor (EGF), hydrocortisone, insulin, isoproterenol, and tri-iodo-thyronine, wherein the medium does not contain any of the following supplements: a TGF-beta inhibitor, a Wnt inhibitor and a fibroblast growth factor; or

ii) for producing early pigmented retinal pigment epithelial (RPE) precursor cells, in a cell culture medium which does not contain any of the following supplements: a TGF-beta inhibitor, a Wnt inhibitor and a fibroblast growth factor.

In further aspects, the present invention provides an eye precursor cell, a corneal epithelial precursor, a corneal epithelial cell, stratified corneal epithelia, an early pigmented RPE precursor, and a mature RPE cell obtainable by the present methods.

Also provided are an induction medium comprising TGF-beta inhibitor, a Wnt inhibitor and a fibroblast growth factor, and uses of various herein-disclosed culture media for producing eye precursor cells, corneal epithelial precursor cells, corneal epithelial cells, stratified corneal epithelium, early pigmented RPE precursor cells, or mature RPE cells, from pluripotent stem cells.

Other aspects, specific embodiments, objects, details, and advantages of the invention are set forth in the dependent claims and will become apparent from the following drawings, detailed description and examples.

### BRIEF DESCRIPTION OF THE DRAWINGS

In the following, invention will be described in greater detail by means of preferred embodiments with reference to the attached drawings, in which

FIG. 1 exemplifies structures of two commercial small molecular TGF-b1-inhibitors (D 4476 and SB 505124).

FIG. 2 exemplifies two commercially available small molecular Wnt-inhibitor structures (IWP-2 and IWP-3).

FIG. 3 gives an overview on the strategy of the experiments conducted to validate the present method. It explains as function of time the two stages of cultivation, i.e. the induction stage and differentiation stage, for producing corneal epithelial (CE) and RPE linages, and points out the key time-points. It also clearly shows the media studied in each test.

FIG. 4 illustrates differentiation results obtained after 4 days in the induction stage, carried out in different culture media. From left to right: sample of undifferentiated stem cells as control (Undiff); commercial medium marketed for cultivation of corneal epithelial cells (CnT-30); RegES medium modified according to the present invention to comprise a TGF-beta inhibitor, a Wnt-inhibitor and a fibroblast growth factor (RegESinduction); unsupplemented RegES medium (RegESbasic). Differentiation was followed measuring two markers, POU5F1, which is present in undifferentiated cells and PAX6, which indicates differentiation into eye specific cell lineages.

FIG. 5 illustrates the outcome of an embodiment of the present differentiation method, wherein human induced pluripotent stem cells (hiPSC) or human embryonic stem cells (hESC) were differentiated into corneal epithelial precursor cells. The maturation stage was carried out either in commercially available CnT-30 medium or SHEM medium containing EGF, hydrocortisone, insulin, isoproterenol and tri-iodo-thyronine. Corresponding results were obtained regardless of the maturation medium and cell type used.

FIG. 6 illustrates differentiation of hiPSCs or hESCs into RPE pre-cursor cells in different culture conditions. Pigmentation rate of RPE cells was assessed qualitatively after a total of 50 days in differentiation culture, and was shown to be notably enhanced by induction with small molecules and bFGF (RPEinduction).

### DETAILED DESCRIPTION OF THE INVENTION

In some aspects, the present invention provides a one-stage induction method for producing eye precursor cells, and a two-stage differentiation method comprising said induction method and a further differentiation and maturation stage for differentiating said eye precursor cells into differentiated eye cells, such as corneal epithelial precursor cells or early pigmented retinal pigment epithelial (RPE) precursor cells, and further into mature corneal epithelial cells or mature RPE cells, respectively. As used herein, the terms “pre-cursor” and “progenitor” may be used interchangeably unless otherwise indicated.

### Induction Method and Medium

The present induction method is aimed at inducing pluripotent stem cells towards surface ectoderm and eye precursor cells by subjecting said stem cells, preferably in a suspension culture, to an induction medium comprising active “induction supplements”, i.e. a TGF-beta inhibitor, a Wnt inhibitor and a fibroblast growth factor. Both said induction method and said induction medium are provided herein.

If the induction method is to be carried out in adherent culture, it is advantageous to use substrates coated with extracellular matrix (ECM) proteins as generally known in the art and discussed in more detail below under “differentiation method and media”.

As used herein, the term “pluripotent stem cell” refers to any stem cell having the potential to differentiate into all cell types of a human or animal body, not including extra-embryonic tissues. These stem cells include both embryonic stem cells (ESCs) and induced pluripotent cells (iPSCs). Hence, the cells suitable for the method of the present invention include stem cells selected from iPSCs and ESCs.

ESCs are of great therapeutic interest because they are capable of indefinite proliferation in culture and are thus capable of supplying cells and tissues for replacement of failing or defective human tissue. Producing eye precursor cells from human embryonic stem cells may meet ethical challenges. According to one embodiment, human embryonic stem cells may be used with the proviso that the method itself or any related acts do not include destruction of human embryos.

Induced pluripotent stem cells, commonly abbreviated as iPS cells or iPSCs are a type of pluripotent stem cell artificially derived from a non-pluripotent cell—typically an adult somatic cell—by inducing a forced expression of specific genes. One benefit of use of iPS cells is avoidance of the involvement embryonic cells altogether, and hence any ethical questions thereof. Therefore, according to another embodiment of the present invention, when producing eye precursor cells, use of iPS cells is preferred.

Induced pluripotent stem cells are similar to natural pluripotent stem cells, such as embryonic stem cells, in many aspects, such as the expression of certain stem cell genes and proteins, chromatin methylation patterns, doubling time, embryoid body formation, teratoma formation, viable chimera formation, and potency and differentiability, but the full extent of their relation to natural pluripotent stem cells is still being assessed. Induced pluripotent cells are typically made from adult skin cells, blood cells, stomach, or liver, although other alternatives may be possible. A man skilled in the art is familiar with research and therapy potential of iPS cells, i.e. from publication of Bilic and Izpiva Belmonte (2012).

As used herein the term “xeno-free” refers to absence of any foreign material or components. Thus, in case of human cell culture, this refers to conditions free from non-human animal components. In other words, when xeno-free conditions are desired for production of eye precursor cells, or any cells maturated therefrom, for human use, ES or iPS cells are selected to be of human origin.

As used herein, the term “eye precursor cell” refers broadly to any cell lineage of the eye induced from pluripotent stem cells using the present induction method and/or induction medium. Eye precursor cells are characterized by down-regulation of the pluripotency marker OCT-4 (also known as POU5F1) and up-regulation of PAX6, a gene indicating differentiation into eye specific cell lineages.

In the present induction method, pluripotent stem cells are cultured in the present induction medium for a period of time which may vary depending on different variables such as the final composition of the induction medium and the purpose of the induction method. Typically, the duration of the induction may vary from a couple of days to several days. A preferred time range is from about four days to about seven days, i.e. from about 96 hours to about 168 hours. As used herein, the term “about” refers to a variation of 10 percent of the value specified. Thus, the term “about four days” carries a variation from 87 to 105 hours, while the term “about seven days” carries a variation from 152 to 186 hours. If an induction time shorter than 4 to 7 days is used, down-regulation of OCT4 and up-regulation of PAX6 is weak leading to less efficient differentiation as determined by weak expression of precursor markers PAX6 and p63 Moreover, if an induction time longer than 4 to 7 days is used, more neural differentiation can be expected, because human pluripotent stem cells have a known tendency to differentiate towards neural lineages, especially in the presence of basic fibroblast growth factor.

As set forth above, the present induction medium comprises a TGF-beta inhibitor, a Wnt inhibitor and a fibroblast growth factor as active supplements. These induction supplements were found to enhance differentiation of pluripotent stem cells towards eye precursor cells and improve their further differentiation efficiency into clinically valuable eye cells, such as corneal epithelial precursor cells, corneal epithelial cells, stratified corneal epithelia, early pigmented RPE cells, or mature RPE cells.

The present induction medium may be considered to consist of or comprise a basal medium and the present induction supplements. However, further supplements common in the art may be applied. As used herein, the term “common cell culture supplements” refers to ingredients used in practically every cell culture medium including antibiotics, L-glutamine, and serum, serum albumin or a serum replacement, preferably a defined serum replacement.

On the other hand, in some embodiments, the induction medium does not contain ingredients other than the induction supplements, basal medium, antibiotics, L-glutamine, and a defined serum replacement. In some further embodiments, the induction supplements are a TGF-beta inhibitor of Formula I or II, a Wnt inhibitor according to Formula III, and bFGF. In a still further embodiment, the induction supplements are SB-505124, IWP-2, and bFGF.

Any of the aforementioned embodiments may form a basis for additional or alternative embodiments, wherein the induction medium does not comprise any supplements generally known to be inductive for differentiation types other than differentiation towards eye lineages, such types as neural differentiation. Such generally known supplements include, but are not limited to, retinoic acid, ascorbic acid, brain-derived neurotrophic factor BDNF, and glial-derived neurotropichic factor GDNF.

The basal medium may be any stem cell culture medium in which stem cells can effectively be differentiated. In a preferred embodiment, the basal medium is RegESbasic medium. In other preferred embodiments, the basal medium is RPEbasic. According to further preferred embodiment, the induction medium is xeno-free, serum-free or defined, more preferably a combination of these and most preferably xeno-free, serum-free and defined at the same time. These terms are defined below.

In a preferred composition of the induction medium, the content of the TGF-beta inhibitor is from 1 μM to 100 μM, preferably from 1 μM to 30 μM, the Wnt-inhibitor is from 1 μM to 100 μM, preferably from 1 μM to 30 μM, and the content of fibroblast growth factor is from 1 ng/ml to about 1000 ng/ml, preferably about 2 ng/ml to about 100 ng/ml, and more preferably about 30 ng/ml to about 80 ng/ml.

### TGF-beta (TGF-13) Inhibitor

As used herein, with “TGF-beta inhibitor” is referred functionally to a substance capable of inhibiting transforming growth factor β1.

Transforming growth factor β1 (TGF-β1) is a member of a large superfamily of pleiotropic cytokines that are involved in many biological activities, including growth, differentiation, migration, cell survival, and adhesion in diseased and normal states. Nearly 30 members have been identified in this superfamily. These are considered to fall into two major branches: TGFb/Activin/Nodal and BMP/GDF (Bone Morphogenetic Protein/Growth and Differentiation Factor). They have very diverse and often complementary functions. Some are expressed only for short periods during embryonic development and/or only in restricted cell types (e.g. anti-Mullerian hormone, AMH, Inhibin) while others are widespread during embryogenesis and in adult tissues (e.g. TGF(β1 and BMP4). TGF-β1 is a potent regulator in the synthesis of the extracellular matrix (fibrotic factor) and plays a role in wound healing.

In chemical and structural terms, suitable TGF-beta inhibitory function may be found among proteins and small organic molecules. A man skilled in the art is aware of means for isolating proteins from biological matrixes or producing them i.e. by recombinant techniques.

Compounds exhibiting TGF-beta inhibitory activity may be found by screening. Preferably a TGF-beta inhibitor is an organic molecule having a relatively low molar mass, e.g a small molecule having molar mass less than 800 g/mol, preferably less than 500 g/mol. As a general structure, Formula I, a suitable low molar mass TGF-inhibitor may be described as:

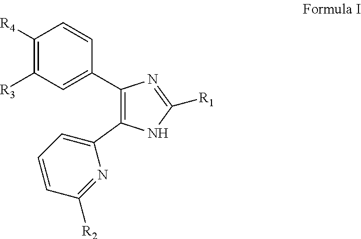

wherein R₁ represents an C₁-C₅ aliphatic alkyl group, carboxylic acid, amide, and R₂ represents an C₁-C₅ aliphatic alkyl, R₃ and R₄ represent aliphatic alkyls including heteroatoms, O or N, which may be linked together to form a 5- or 6 member heteroring.

A typical structure comprises a hetero ring having 2 oxygen atoms, when it can be referred to as a small molecule of general formula II:

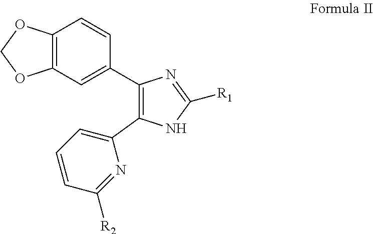

wherein, R₁ represents an C₁-C₅ aliphatic alkyl group, an aromatic carboxylic acid or amide, and R₂ represents an C₁-C₅ aliphatic alkyl.

One example of such an TGF-b1-inhibitor is 4-[4-(1,3-Benzodioxol-5-yl)-5-(2-pyridinyl)-1H-imidazol-2-yl]-benzamide; 4-[4-(3,4-Methylenedioxyphenyl)-5-(2-pyridyl)-1H-imidazol-2-yl]-benzamide; 4-(5-benzol[1,3]dioxol-5-yl-4-pyridin-2-yl-1H-imidazol-2-yl)-benzamide hydrate, with the chemical formula in FIG. 1, which is commercially available from suppliers and marketed as a selective inhibitor of transforming growth factor-β type I receptor (ALK5), ALK4 and. Selectively inhibits signaling from TGF-β and activin; does not inhibit other ALK family members. Another example of TGF-inhibitors is 2-(5-benzo[1,3]dioxol-5-yl-2-tert-butyl-3H-imidazol-4-yl)-6-methylpyridine hydrochloride hydrate, the structure of which is given in FIG. 1 as well.

However, other small molecules exhibiting TGF-beta inhibitory activity or commercially marketed as TGF-inhibitors may be equally suitable in the context of the present invention. When selecting said TGF-beta inhibitor from substances obtainable by chemical synthesis or recombinant production, a defined medium can be provided. It also complies with requirements of xeno-free and serum-free conditions.

### Wnt Inhibitor

As used herein, a “Wnt inhibitor” refers to a substance capable of inhibiting Wnt signaling pathway. More specifically Wnt-inhibiting functions relate to i.e. preventing palmitylation of Wnt proteins by porcupine (Porc), a membrane-bound O-acyltransferase, thereby blocking Wnt secretion and activity. A Wnt inhibitor also blocks phosphorylation of the Lrp6 receptor and accumulation of both Dvl2 and β-catenin1. The inhibition of the Wnt pathway through the use of a Wnt inhibitor has also been shown to promote the formation of cardiomyocytes from human embryonic stem cell-derived mesodermal cells.

Both protein and small molecular Wnt inhibitors are known in the art. In context of the present invention, small molecular Wnt inhibitors are preferred.

When selecting said Wnt inhibitor from substances obtainable by chemical synthesis, it contributes to providing a defined medium. Suitable compounds have been discovered by screening a group of organic molecules. Preferably such an inhibitor is an organic molecule having a relatively low molar mass, e.g. a small molecule having molar mass less than 800 g/mol, preferably less than 500 g/mol. As a general structure, Formula III, a suitable small molecular Wnt inhibitor may be described as:

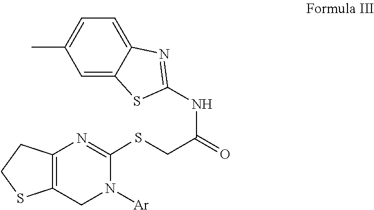

wherein Ar refers to substituted or unsubstituted aryl group.

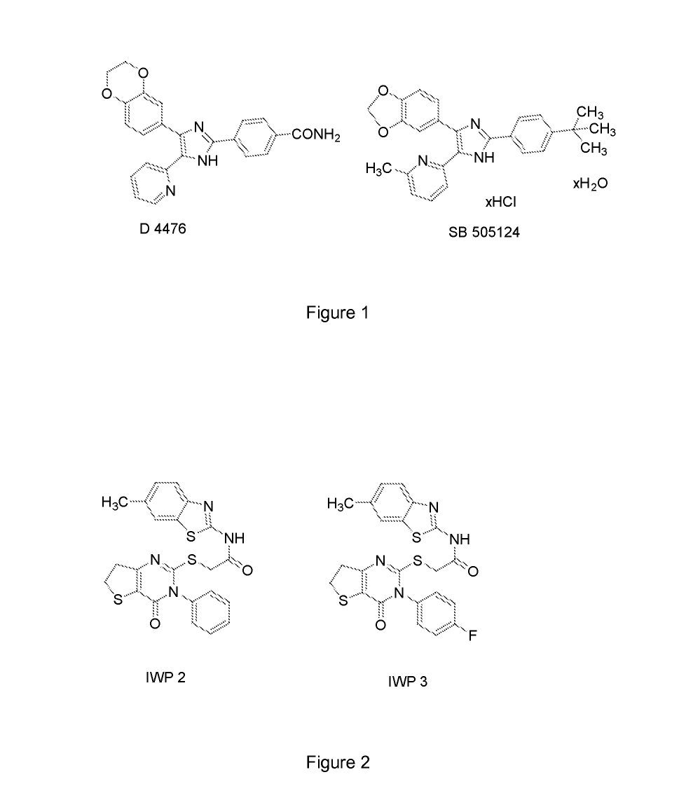

Commercially known Wnt inhibitors are exemplified in FIG. 2.

### Fibroblast Growth Factor

In the medium of the present invention, a fibroblast growth factor is required to contribute to the differentiation. Fibroblast growth factors, or FGFs, are a family of growth factors generally involved in angiogenesis, wound healing, and embryonic development. The FGFs are heparin-binding proteins and interactions with cell-surface-associated heparan sulfate proteoglycans have been shown to be essential for FGF signal transduction. FGFs are key players in the processes of proliferation and differentiation of wide variety of cells and tissues.

Fibroblast growth factors suitable for use in the present invention include fibroblast growth factors (FGFs) such as basic FGF (bFGF or FGF-2). While FGF is preferably used, other materials, such as certain synthetic small peptides (e.g. produced by recombinant DNA variants or mutants) designed to activate fibroblast growth factor receptors, may be used instead of FGF. Fibroblast growth factors may be included in the serum replacement used as basal medium or they may be added separately to the final cell culture medium according to the present invention.

### Differentiation Method and Media

In some embodiments, the present differentiation method is a two-stage method comprising the above-described induction method, preferably but not necessarily carried out in a suspension culture, followed by a differentiation stage in adherent culture. In the latter stage, eye precursor cells produced in the induction stage are differentiated into eye lineages, such as corneal epithelial precursor cells or early pigmented RPE cells.

In some other embodiments, the present differentiation method is a three-stage method comprising the above-described induction method, preferably but not necessarily carried out in suspension culture, followed by a differentiation stage and a further maturation stage in adherent culture. In the maturation stage, corneal epithelial precursor cells are maturated into mature corneal epithelial cells or even into corneal stratified epithelia, while early pigmented RPE precursors produced in the differentiation stage are maturated into mature RPE cells.

In practice, the differentiation stage and maturation stage are carried out successively in the same way; they only differ from each other in respect of timing, as explained in more detail below. Even the culture medium to be used in these stages is the same. Thus, the terms “differentiation medium” and “maturation medium” are interchangeable. The same applies to the terms “differentiation supplements” and “maturation supplements”. If desired, a shift from a differentiation stage to a maturation stage may concern only a selected subpopulation of cells obtained from the differentiation stage.

Preferably, the differentiation and maturation conditions are xeno-free, serum-free or defined, more preferably a combination of these, and most preferably xeno-free, serum-free and defined at the same time.

Since the differentiation and maturation stages are to be performed in an adherent culture and the ability to attach to extracellular matrix (ECM) is considered to be important for epithelial cells, it is advantageous to use substrates, such as cell culture plates or bottles, coated with ECM proteins as generally known in the art. In fact, adhesion to collagen IV is generally used for selecting epithelial precursor cells from limbal epithelial cell populations and differentiating pluripotent stem cells. Thus, in some preferred embodiments, the cell culture substrate is coated with collagen IV. Other non-limiting examples of suitable coating materials include collagen I, laminin, vitronectin, fibronectin, and Matrigel™. When xeno-free conditions are desired for human use, the substrate is to be coated with an ECM protein of human origin. Means and methods for coating cell culture substrates are generally available in the art.

### Corneal Epithelial Differentiation

In some embodiments of the present invention, the eye precursor cells obtainable by the present induction method are differentiated further into corneal epithelial precursor cells. This embodiment may be termed as a two-stage differentiation method.

In this context, the term “corneal epithelial precursor cells” refers to cells which are positive for, i.e. express, corneal epithelial marker p63, which may be quantified with the help of immunofluorecent staining. According to an embodiment, the corneal epithelial precursor cells expressing said marker p63 represent at least 65%, preferably at least 75%, most preferably at least 90% of the total cell population. Population of corneal epithelial precursor cells may be used clinically.

Typically, obtaining corneal epithelial precursor cells requires culturing eye precursor cells under the present corneal differentiation conditions for about 10 to about 35 days, preferably for about 25 days. In some preferred embodiments, corneal epithelial precursor cells are obtained by carrying out the induction stage for about four to about seven days followed by the corneal differentiation stage for about 23 to 26 days. The most pure p63-positive cell population can be obtained after a differentiation stage of this length. Shorter differentiation time yields more heterogeneous cell populations, while longer differentiation time results in terminal maturation towards corneal epithelial cells.

In some further embodiments, said corneal epithelial precursor cells may be maturated even further into mature corneal epithelial cells or stratified corneal epithelia, as demonstrated by a characteristic marker expression and morphology. Such further maturation may be obtained by continued cell culturing, typically for an additional 10 to 20 days, in the present corneal maturation conditions, which in practice correspond to the corneal differentiation conditions. This embodiment may be termed as a three-stage differentiation method.

A suitable culture medium for use in the differentiation and maturation stages may be, for instance, any available corneal medium such as CnT-30 which is commercially available from CELLnTECH, or any supplemental hormonal epithelial medium (SHEM) suitable for culturing corneal epithelial cells. In some other embodiments, the differentiation and maturation medium may be composed by adding ingredients such as one or more differentiation and maturation supplements selected from the group consisting of epidermal growth factor (EGF), hydrocortisone, insulin, isoproterenol and tri-iodo-thyronine, into any suitable basal medium. In some embodiments, the corneal differentiation and maturation medium does not contain ingredients other than said one or more differentiation and maturation supplements, basal medium, antibiotics, L-glutamine, and a defined serum replacement. In some further embodiments, the basal medium is a 1:1 mixture of DMEM and Ham's F12 nutrient mixture.

Any of the aforementioned embodiments may form a basis for additional or alternative embodiments, wherein the differentiation and maturation medium does not comprise any supplements, which are generally known to cause differentiation towards cells types other than eye cells, such types as neural differentiation. Such generally known supplements include, but are not limited to, retinoic acid, ascorbic acid, brain-derived neurotrophic factor BDNF, and glial-derived neurotropichic factor GDNF.

Importantly, differentiation of pluripotent stem cells into corneal epithelial precursor cells and further maturation into corneal epithelial cells, or stratified corneal epithelia was significantly better when the cells were differentiated by the present induction method followed by the present corneal differentiation and maturation stage than when performing the corneal differentiation and maturation stage without prior induction. Improved results were obtained e.g. regarding better adhesion to collagen IV-coated substrates, uniform corneal epithelial cell morphology, and enhanced expression of p63, as well as other key markers (most notably cytokeratins 3, 12 and 15) both on the gene and protein level.

### Retinal Pigment Epithelium Differentiation

In some embodiments of the present invention, the eye precursor cells obtainable by the present induction method are differentiated further into early pigmented RPE precursor cells. This embodiment may be termed as a two-stage differentiation method.

As used herein, the term “early pigmented RPE precursors” refers to cells having early pigmentation (melanogenesis), expression of specific genes such as MITF and PMEL, and further capability to differentiate to fully maturated RPE cells having hexagonal and cobblestone-like RPE cell morphology, forming highly polarized epithelium and expressing mature RPE-related proteins such as Bestrophin, CRALBP, MERTK and RPE65.

Typically, obtaining early pigmented RPE precursor cells takes a minimum of about 20 days of cell culturing in the present RPE differentiation conditions. In some preferred embodiments, early pigmented RPE cells are obtained by carrying out the induction stage for about four to about seven days followed by the RPE differentiation stage for about 40 days. Shorter differentiation time yields more heterogeneous cell populations, while longer differentiation time results in terminal maturation towards RPE cells.

In some further embodiments, said early pigmented RPE precursors may be maturated even further into mature RPE cells. Such further maturation may be obtained by continued cell culturing of selected pigmented cell populations, typically for about 50 to about 120 days, preferably for about 80 days, under the present RPE maturation conditions, which in practice correspond to the RPE differentiation conditions. This embodiment may be termed as a three-stage differentiation method.

Differentiation of eye precursors into early pigmented RPE precursors may be obtained simply by withdrawing the induction supplements from the culture medium. In practice, this may be achieved by replacing the induction medium with a differentiation medium which does not contain the induction supplements. In an embodiment, such a differentiation medium is RPEbasic, the composition of which is given in the experimental part. A conventional RPE differentiation method is based on spontaneous differentiation of pluripotent cells upon removal of bFGF (Vaajasaari et al. 2011). However, better differentiation and maturation rate was obtained by the present differentiation method, which included the induction stage.

Although withdrawal of the induction supplements resulted in a good RPE differentiation and maturation rate, even better rates were obtained when the withdrawal was combined with the use of one or more of the following RPE differentiation supplements: taurine, hydrocortisone, tri-iodo-thyronine, activin A, insulin, transferrin, sodium selenite, putrescine, and progesterone. In some embodiments, the RPE differentiation and maturation medium does not contain ingredients other than said one or more RPE differentiation and maturation supplements, basal medium, antibiotics, L-glutamine, and a defined serum replacement. In some further embodiments, the basal medium is a MEM alpha medium. A non-limiting example of a known cell culture medium suitable for use as an RPE differentiation and maturation medium in the differentiation and maturation stage of the present differentiation method is feRPE medium, originally developed for culturing isolated human primary RPE cells (Maminishkis et al. 2006).

Significantly better RPE differentiation and maturation rate was obtained by the present differentiation method, regardless of whether it was applied as two-stage or three-stage method, as compared with that obtained after an induction lacking the induction supplements (RPEbasic).

### General Features of Present Media

The culture medium may be considered to consist of basal medium and supplements. In the present induction medium, the three essential supplements are TGF-beta inhibitor, a Wnt inhibitor and a fibroblast growth factor. In the present corneal epithelial differentiation and maturation medium, exemplary or preferred supplements additional to common cell culture supplements are selected from the group consisting of EGF, hydrocortisone, insulin, isoproterenol, and tri-iodo-thyronine. In the present RPE differentiation and maturation medium, exemplary or preferred supplements additional to common cell culture supplements are selected from the group consisting of taurine, hydrocortisone, tri-iodo-thyronine, activin A, insulin, transferrin, sodium selenite, putrescine, and progesterone. However, in the context culture media, further supplements common in the art may be applied, unless they are known to direct differentiation towards tissues other than the eye. When referring to components of a medium, the term includes both supplements and ingredients to the basal medium.

When in use or when ready for use, the present culture media comprise appropriate essential supplements set forth above. However, according to common practice in the field, the ingredients for a medium may be provided as a concentrate comprising said components or a set of vials from which suitable combination is prepared in a laboratory according to instructions provided.

As referred here, “culture medium” or “cultivation medium” refers broadly to any liquid or gel formulation designed to support the growth of microorganisms, cells or small plants. When referring to formulation designed for cell maintenance and growth, term “cell culture medium” is used. In the art, expressions such as induction medium, growth medium, differentiation medium, maturation medium etc. can be considered as subspecies to general expression culture medium. A man skilled in the art is familiar with the basic components necessary to maintain and nourish the living subjects in or on the culture medium, and commercial basic media are widely available. Typically such basic components are referred to as “basal medium”, which contains necessary amino acids, minerals, vitamins and organic compounds. Generally, it can be obtained from biological sources, such as serum or be combined from isolated and pure ingredients. If desired, basal medium may be supplemented with substances contributing to special features or functions of culture medium. Very common supplements include antibiotics, which are used to limit growth of contaminants.

Non-limiting examples of suitable basal media to be used in the present cell culture media of various embodiments include KnockOut Dulbecco's Modified Eagle's Medium (KO-DMEM), Dulbecco's Modified Eagle's Medium (DMEM), Minimal Essential Medium (MEM), Basal Medium Eagle (BME), RPMI 1640, F-10, F-12, Glasgow's Minimal Essential Medium (G-MEM), Iscove's Modified Dulbecco's Medium and any combinations thereof. In some preferred embodiments, RegES medium altered by omitting retinol, bFGF and activin A (referred to herein as RegESbasic) is used as a basal medium. In some more preferred embodiments, RegESbasic is used as a basal medium in the present induction medium.

For better clinical acceptance all culture media used herein are preferably substantially xeno-free, substantially serum-free or defined, more preferably combinations of these and most preferably xeno-free, serum-free and defined at the same time. With substantially is meant here that unintentional traces are irrelevant and what is under clinical or laboratory regulations considered and accepted as xeno-free, serum-free or defined, applies here as well.

Traditionally, serum, especially fetal bovine serum (FBS) has been valued in cell cultures providing essential growth and survival components for in vitro cell culture of eukaryotic cells. It is produced from blood collected at commercial slaughterhouses from cattle bred to supply meat destined for human consumption. “Serum free” indicates that the culture medium contains no serum, either animal or human. Defined medium is valuated when there are contradictions for use of undefined media, e.g. “conditioned medium”, which refers to spent media harvested from cultured cells containing metabolites, growth factors, and extracellular matrix proteins secreted into the medium by the cultured cells. Undefined media may be subject to considerable dissimilarities due to natural variation in biology. Undefined components in a cell culture compromise the repeatability of cell model experiments e.g. in drug discovery and toxicology studies. Hence, “defined medium” or “defined culture medium” refers to a composition, wherein the medium has known quantities of all ingredients. Typically, serum that would normally be added to culture medium for cell culture is replaced by known quantities of serum components, such as, e.g., albumin, insulin, transferrin and possibly specific growth factors (i.e., basic fibroblast growth factor, transforming growth factor or platelet-derived growth factor).

A chemically defined medium is a growth medium suitable for the in vitro cell culture of human or animal cells in which all of the chemical components are known. A chemically defined medium is entirely free of animal-derived components and represents the purest and most consistent cell culture environment. By definition chemically defined media cannot contain fetal bovine serum, bovine serum albumin or human serum albumin as these products are derived from bovine or human sources and contain complex mixes of albumins and lipids.

Chemically defined media differ from serum-free media in that bovine serum albumin or human serum albumin with either a chemically defined recombinant version (which lacks the albumin associated lipids) or synthetic chemical such as the polymer polyvinyl alcohol which can reproduce some of the functions of BSA/HSA. The next level of defined media, below chemically defined media is protein-free media. These media contain animal protein hydrolysates and are complex to formulate although are commonly used for insect or CHO cell culture.

According to some embodiments, the present media comprises a xeno-free serum replacement formulation. A defined xeno-free serum replacement formulation or composition may be used to supplement any suitable basal medium for use in the in vitro derivation, maintenance, proliferation, or differentiation of stem cells. Said serum replacement may be used to supplement either serum-free or serum-containing basal mediums, or any combinations thereof. When xeno-free basal medium is supplemented with the present xeno-free serum replacement, the final culture medium is xeno-free as well. One example is described in Rajala et al. 2010 which is incorporated here as reference describing a xeno-free serum replacement applicable in the context of the present invention. Another non-limiting example of a defined serum replacement is KnockOut™ Serum Replacement (Ko-SR) commercially available from Life Technologies.

A man skilled in the art is familiar with different culture medium concentrations. Often, culture medium is diluted and prepared to the final composition immediately before use. Therefore, it is understood that any stock solution or preparation kit suitable for use in such immediate preparation is included in the scope of the present invention as well. For example, for preparation of a culture medium according to present description, a cell culture kit comprises the TGF-beta inhibitor, the Wnt-inhibitor and the fibroblast growth factor each in a separate container or any combinations thereof as supplements, and optionally other components, such as basal medium or supplies for preparation thereof, as well.

### Experimental Part

### Materials and Methods

### Cell Line and Cell Culture Media

Undifferentiated pluripotent stem cell line was derived and characterized as described previously (Skottman 2010). The cell line was cultured on mitotically inactivated human foreskin fibroblast (hFF) feeder cells (CRL-2429, ATCC). Undifferentiated pluripotent stem cells were maintained in a culture medium consisting of Knock-Out Dulbecco's Modified Eagle Medium (KO-DMEM) supplemented with 20% Knock-Out Serum Replacement (KO-SR), 2 mM GlutaMax-I, 0.1 mM 2-mercaptoethanol (all from Invitrogen), 1% non-essential amino acids (NEAA), 50 U/ml penicillin/streptomycin (both from Lonza Group Ltd) and 8 ng/ml human basic fibroblast growth factor (bFGF, Pepro-Tech). The culture medium was changed five times a week and undifferentiated colonies were enzymatically passaged onto fresh feeder cell layers at ten-day intervals.

Three different media were used for corneal epithelial differentiation: A) Commercially-available defined and serum-free CnT-30 corneal epithelium medium (CELLnTEC Advanced Cell Systems) supplemented with the appropriate supplements provided with the medium and 50 U/ml penicillin/streptomycin. B) Serum-free and xeno-free RegES medium originally developed for the culture of undifferentiated stem cells (Rajala et al. 2010). The medium composition was altered by omitting retinol, bFGF and activin A, and the resulting differentiation medium is referred to as RegESbasic medium. The composition of said RegESbasic medium was as given in Table 1. C) RegESbasic medium supplemented with 10 μM TGF-beta inhibitor (SB-505124 Sigma), 10 μM Wnt-inhibitor (IWP-2 Merck Biosciences) and 50 ng/ml bFGF and the resulting induction medium is referred to as RegESinduction medium.

TABLE 1
RegESbasic medium representing an example of xeno-free cell
culture medium according to Rajala et al., 2010.

| | | |
|---|---|---|
| Component | Concentration (mg/L) | Manufacturer |

Amino acids

| | | |
|---|---|---|
| Glycine | 53 | Sigma |
| L-histidine | 183 | Sigma |
| L-isoleucine | 615 | Sigma |
| L-methionine | 44 | Sigma |
| L-phenylalanine | 336 | Sigma |
| L-proline | 600 | Sigma |
| L-hydroxyproline | 15 | Sigma |
| L-serine | 162 | Sigma |
| L-threonine | 425 | Sigma |
| L-tryptophan | 82 | Sigma |
| L-tyrosine | 84 | Sigma |
| L-valine | 454 | Sigma |

Vitamins, antioxidants and trace elements

| | | |
|---|---|---|
| Ascorbic acid | 50 | Sigma |
| Glutathione | 1.5 | Sigma |
| Selenium | 1 × 10⁻⁵ | Sigma |
| Thiamine | 9 | Sigma |
| Trace elements B | 1:1000 | Cellgro |
| Trace elements C | 1:1000 | Cellgro |

Proteins

| | | |
|---|---|---|
| Human serum albumin* | 10 000 | Sigma |
| Insulin | 100 | Invitrogen |
| Transferrin | 8 | Sigma |

Other components

| | | |
|---|---|---|
| NEAA (100x) | 1% | Lonza |
| Glutamax-I | 2 mM | Invitrogen |
| β-mercaptoethanol | 0.1 mM | Invitrogen |

Basal medium: KO-DMEM (Invitrogen)
*To provide better consistency and less variation, HSA can be substituted with recombinant HSA.

For RPE differentiation, three cell culture media were used. RPEbasic medium consisted of KO-DMEM supplemented with 20% KO-SR, 2 mM GlutaMax-I, 0.1 mM 2-mercaptoethanol, 1% NEAA, and 50 U/ml penicillin/streptomycin. For eye precursor induction, this medium was supplemented with 10 μM SB-505124, 10 μM IWP-2 and 50 ng/ml bFGF (RPEinduction). Further RPE differentiation and maturation was carried out either in RPEbasic medium, or feRPE medium consisting of Minimal Essential Medium (MEM), alpha modification (Sigma-Aldrich), supplemented with 15% KO-SR, 2 mM GlutaMax-I, 1% NEAA, 1% N1 medium supplement (Sigma-Aldrich), 0.25 mg/ml taurine, 20 ng/ml hydrocortisone, 0.013 ng/ml tri-iodo-thyronine, and 50 U/ml penicillin/streptomycin. N1 supplement consisted of 0.5 mg/ml recombinant human isulin, 0.5 mg/ml human transferrin (partially iron-saturated), 0.5 μg/ml sodium selenite, 1.6 mg/ml putrescine, and 0.73 μg/ml progesterone.

### Eye Precursor Induction

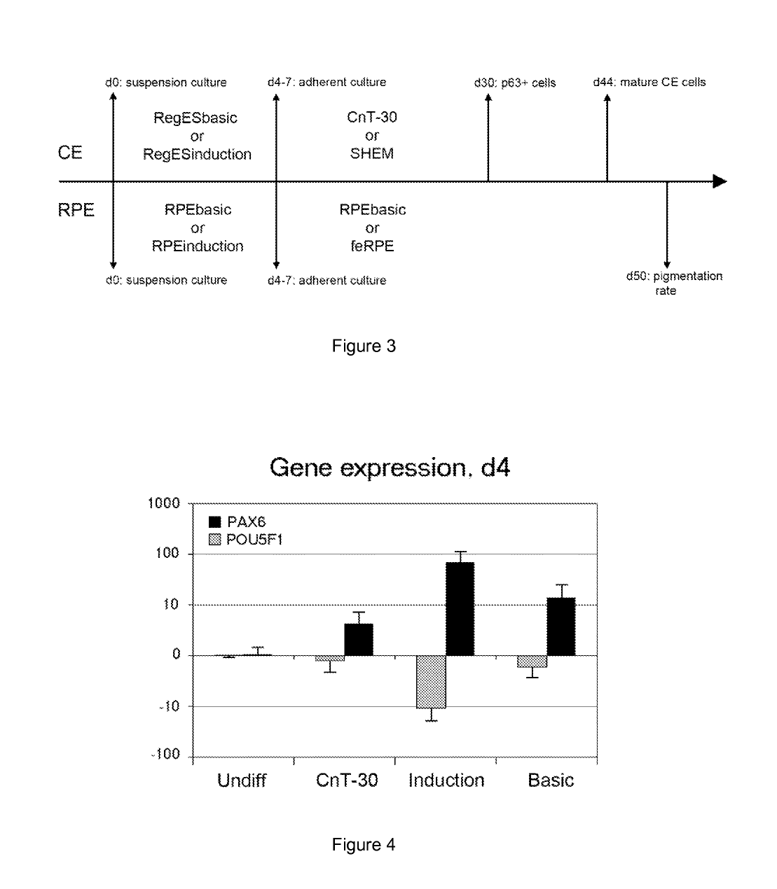

The experimental design is schematically presented in FIG. 3. To initiate eye precursor differentiation of pluripotent stem cells, undifferentiated colonies were manually dissected and transferred to suspension culture in 6-well plates (Corning ultra-low attachment). Cells were cultured as three-dimensional cell clusters for four to seven days, changing the culture medium daily. This induction stage was carried out either in RPEinduction (RPE differentiation) or RegESinduction (cornea epithelial differentiation) and in compared control conditions without inductive molecules. It would have also been possible to perform the induction by using RPEinduction for corneal epithelial differentiation and RegESinduction for RPE differentiation.

### Corneal Epithelial Differentiation and Maturation

After the induction stage, cell clusters were plated onto cell culture substrate coated with human placental collagen IV (5 μg/cm², Sigma) at a density of about 50 clusters/cm². Either 24-well plates (Corning CellBIND) or 24-well hanging cell culture inserts (Millipore, 1 μm pore size) were used. Cells were maintained in adherent culture for 40 more days in either CnT-30 medium (conditions A-C) or RegESbasic medium (control), replacing the culture medium three times a week. Alternatively, CnT-30 medium was replaced with supplemental hormonal epithelial medium (SHEM), consisting of DMEM/F12 basal medium supplemented with 15% KO-SR, 2 mM GlutaMax-I, 100 μg/ml hydrocortisone (Sigma), 10 μg/ml insulin (Invitrogen), 0.25 μg/ml isoproterenol (Sigma), 1.35 ng/ml tri-iodo-thyronine (Sigma), 10 ng/ml EGF (PeproTech) and 50 U/ml penicillin/streptomycin.

### RPE Differentiation and Maturation

After the induction stage, cell clusters were plated onto cell culture substrate coated with human placental collagen IV (5 μg/cm², Sigma) at a density of about 50 clusters/cm². Cells were maintained in adherent culture for at least 45 more days in either RPEbasic or feRPE medium, replacing the culture medium three times a week. Appearance of the first pigmented areas was followed daily. Moreover, abundance of pigmentation was visually compared between culture conditions after a total of 50 days in differentiation culture.

After a total of 50 days in differentiation culture, pigmented areas were selected, manually dissected, and re-plated onto new well-plates coated with collagen IV. The purified RPE cell populations were then further maturated using either RPEbasic or feRPE medium.

Quantitative Real Time Polymerase Chain Reaction (qPCR)

Total RNA was extracted from undifferentiated pluripotent stem cells, and from cell aggregates collected after the induction phase, using NucleoSpin RNA II kit (Macherey-Nagel, GmbH & Co). The RNA concentration of each sample was determined using the NanoDrop-1000 spectrophotometer (NanoDrop Technologies). From each RNA sample, 200 ng were used to synthesize cDNA using the high-capacity cDNA RT kit (Applied Biosystems). The resulting cDNA samples were analyzed with qPCR using sequence-specific TaqMan Gene Expression Assays (Applied Biosystems) for POU5F1 (Hs00999632_g1) and PAX6 (Hs01088112_m1). All samples and controls were run as triplicate reactions with the 7300 Real-time PCR system. Results were analyzed with the 7300 System SDS Software (Applied Biosystems) and Microsoft Excel. Based on the cycle threshold (CT) values given by the software, the relative quantification of each gene was calculated by applying the—2ΔΔCt method. Results were normalized to GAPDH (Hs99999905_m1), with the undifferentiated pluripotent stem cells as the calibrator to determine the relative quantities (RQ) of gene expression in each sample. The analysis was repeated four times.

### Immunofluorescence

To assess corneal epithelial differentiation, protein expression of p63 was analyzed and quantified at three time-points (10, 20 and 30 days in differentiation culture) from cells cultured on collagen IV-coated hanging inserts. Cells were fixed with 4% paraformaldehyde (Sigma) for 20 minutes and cell membranes permeabilized with 0.1% Triton-X-100 for 10 minutes. Non-specific binding sites were blocked with 3% BSA for 1 hour. The goat anti-p63 primary antibody (Santa Cruz) was diluted 1:100 in 0.5% BSA, and incubated with the cells for 1 hour at room temperature, or overnight at +4. Primary antibody detection was done with Alexa Fluor 568-conjugated donkey anti-goat secondary antibody (Molecular Probes) diluted 1:800 in 0.5% BSA for 1 hour at room temperature. Samples were mounted onto object glasses in VectaShield mounting medium containing DAPI for visualization of nuclei. Images were captured with fluorescence microscope from multiple randomly selected areas. Total cell numbers were determined by counting the number of DAPI-stained nuclei, and percentages of p63-positive nuclei were consequently quantified. At the end-point of the study, a total of 44 days in differentiation culture, expression of several proteins was analyzed with immunofluorescence. The membranes of hanging cell culture inserts were cut into several pieces and treated in the way described above. Binding of the primary antibodies used was visualized using secondary antibodies against the appropriate species labeled with Alexa Fluor 488 or 568.

Cells cultured on 24-well plates were used for quantitative immuno-fluorescence analysis at the end-point of the study. Cells were rinsed with PBS and detached using TrypLE Select (Gibco) for 5 minutes at +37. Cell suspensions were strained through 40 μm cell strainers and centrifuged at 1500 rpm for 5 minutes. Cell pellets were resuspended in cold PBS, and single-cell suspension volumes adjusted to contain 50 000 cells/150 μl sample. Cells were then spun onto object glasses using a cytocentrifuge (CellSpin II). Immunofluorescence stainings were performed for the resulting cytospin samples as described above. Images of multiple randomly selected areas were captured with a fluorescence microscope. Percentages of cells positive for each marker were quantified in relation to DAPI-stained cells. This analysis was repeated three times.

### Results

### Inhibition of the TGF-13 and Wnt Signaling Pathways Directs Pluripotent Stem Cell Differentiation Towards Eye Specific Lineages

To study the effects of induction medium on early-stage differentiation, expression of POU5F1, a marker of undifferentiated cells, and PAX6, a gene important for eye development, was studied using qPCR. Results are shown in FIG. 4. After the four-day induction phase in suspension culture, expression of POU5F1 decreased in all conditions, while expression of PAX6 increased. The differences in expression levels compared to undifferentiated cells were significantly greater (p<0.05, determined with Mann-Whitney U tests) in cells that underwent induction with SB-505124, IWP-2 and bFGF, than in the other induction media, suggesting higher extent of differentiation in these conditions.

### Small Molecule Induction Increases Corneal Epithelial Differentiation Efficiency and Reproducibility

Protein expression of the commonly-used corneal epithelial progenifor marker p63 was analyzed at ten-day intervals by means of immunofluorescence staining. The first time-point was after six days in adherent culture, a total of ten days in differentiation culture, giving the cell clusters time to properly adhere to the collagen IV-coated substrate. The next time-point was at day 20, and the last one at day 30. Expression of p63 was not detected in cells cultured under spontaneous differentiation conditions in RegESbasic throughout the course of the study, suggesting that corneal epithelial precursor cell differentiation does not take place spontaneously. Cells maintained in CnT-30 medium expressed p63 at each time-point, and expression levels were clearly affected by the induction medium. In order to quantify the differences between the culture conditions, amounts of p63-positive cells were counted at each time-point. After induction with CnT-30, p63 expression was quite varied between biological replicates, masking the overall differences between time-points. In contrast, after induction with RegESinduction medium, the number of p63-positive cells increased with time from about 50% at day 10, to about 90% at day 30, with the least interreplicate variation of all the studied conditions. The corneal precursor cell population after 30 culture demonstrated thus interestingly high degree of differentiation. Induction with RegESbasic medium resulted in the lowest p63 expression levels—on average 25-50% throughout the course of the study, with fairly high variation between replicates.

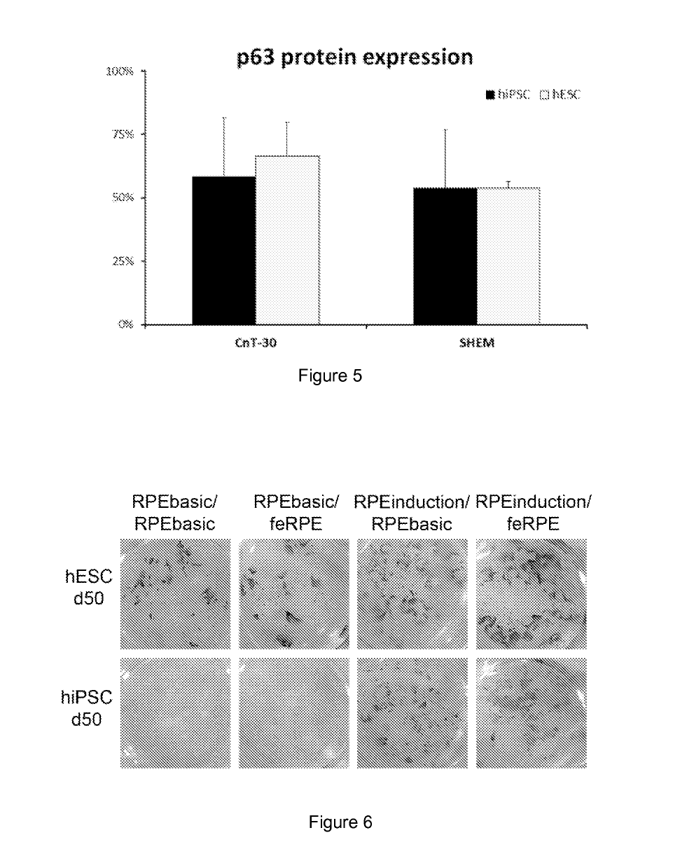

Maturation in SHEM yielded comparable amounts of p63-positive cells after a total of 20 days in differentiation culture, as verified by immunofluorescence staining (FIG. 5).

Corneal Epithelial Cells Can be Differentiated from Pluripotent Stem Cells

Because induction medium affected the subsequent yield of p63-positive corneal epithelial precursor cells, expression of several proteins typical to the corneal precursors (p63 and CK15) and mature epithelium (CK3 and CK12) was quantified also at the end-point of the study.

After a total of 44 days in differentiation culture, cells were analyzed with immunofluorescence, staining positive for progenitor markers p63 and CK15, and markers specific to corneal epithelium, CK3 and CK12. The results showing percentages of differentiation are compiled in Table 2, where ratio between differentiated cells and total number of cells is given, the scales indicating that 0% means that none of the cells (of the population studied) express the differentiation marker, and 100% means that all of the cells express the differentiation marker. The two markers of mature corneal epithelium were detected especially in stratified regions, suggesting that stratification is required for full maturation of corneal epithelium. Expression of progenitor markers and mature markers was for the most part mutually exclusive.

TABLE 2
Results depicting differentiation efficiency
(44 days in differentiation culture).

| | | Expression at the end-point of the |
|---|---|---|
| Marker | Cell type | study |
| p63 | Corneal epithelial precursor | 70% (stDev 4%) |
| CK15 | cells | 60% (stDev 11%) |
| CK3 | Mature corneal epithelial | 35% (stDev 25%) |
| CK12 | cells | 70% (stDev 5%) |

From these results, it can be concluded, that the present method for culturing corneal epithelial precursor cells or mature corneal epithelial cells, comprising culturing stem cells in a culture medium comprising a TGF-beta inhibitor, a Wnt-inhibitor and a fibroblast growth factor as supplements has shown interesting potential.

According to one embodiment said method is characterized by expression of corneal epithelial marker p63, preferably with at least 65% and more preferably at least 70% of cells of the population positive with said marker. According to another embodiment said method is characterized by expression of corneal epithelial marker CK15, preferably with at least 50% and more preferably at least 70% of cells of the population positive with said marker. According to a further embodiment, said method is characterized by simultaneous expression of corneal epithelial marker p63, preferably with at least 65% and more preferably at least 70%, and corneal epithelial marker CK15, preferably with at least 50% and more preferably at least 70% of cells of the population positive with said marker.

According to another embodiment said method is characterized by expression of corneal epithelial marker CK3, preferably with at least 50% and more preferably at least 60% of cells of the population positive with said marker. According to yet another embodiment said method is characterized by expression of corneal epithelial marker CK12, preferably with at least 65% and more preferably at least 75% of cells of the population positive with said marker. According to a further embodiment, said method is characterized by simultaneous expression of corneal epithelial marker CK3, preferably with at least 50% and more preferably at least 60%, and corneal epithelial marker CK12, preferably with at least 65% and more preferably at least 75% of cells of the population positive with said marker.

However, these results representing the end point of the present study, together with results describing p63-expression throughout the course of the study, indicate that it is within competence of a man skilled in the art to determine the time point providing desired enriched population, whether eye precursor cells, corneal epithelium precursors or corneal epithelial cells.

Hence, the present culture medium may be used for producing eye precursor cells or population thereof, corneal epithelial precursor cells or population thereof, corneal epithelial like cells or population thereof, or stratified corneal epithelium from pluripotent stem cells.

### Small Molecule Induction Improves the Rate of RPE Differentiation

After plating three-dimensional cell aggregates, obtained from the induction stage, onto collagen IV-coated substrate, cell attachment and migration were monitored and compared between different culture conditions. Also, appearance of first pigmented cells and pigmentation rate was followed. All these features were notably enhanced after induction (RPEinduction) with the two small molecules and bFGF. In other words, cell attachment and migration were enhanced, and pigmentation onset and rate was slightly improved (Table 3). Pigmentation rate was assessed qualitatively after a total of 50 days in differentiation culture, and was shown to be notably enhanced by small molecule induction, as compared to induction with RPEbasic (FIG. 6). Further maturation of RPE cells could be conducted both in RPEbasic medium and feRPE medium but the maturation in feRPE medium was generally superior to RPEbasic, suggesting that its composition is more optimal for RPE differentiation. All results are summarized in Table 3.

TABLE 3
Differences between culture conditions during RPE differentiation.

| | RPEbasic/ | RPEbasic/ | RPEinduction/ | RPEinduction/ |
|---|---|---|---|---|
| | RPEbasic | feRPE | RPEbasic | feRPE |
| Cell attachment | + | ++ | +++ | +++ |
| Cell migration | + | ++ | +++ | +++ |
| Pigmentation | + | ++ | ++ | +++ |
| onset | | | | |
| Pigmentation | + | + | ++ | +++ |
| rate | | | | |

All references cited are included herein by reference.
It will be obvious to a person skilled in the art that, as the technology advances, the inventive concept can be implemented in various ways. The invention and its embodiments are not limited to the examples described above but may vary within the scope of the claims.

### REFERENCES

Ahmad S, Stewart R, Yung S, Kolli S, Armstrong L, Stojkovic M, Figueiredo F & Lako M (2007): Differentiation of human embryonic stem cells into corneal epithelial-like cells by in vitro replication of the corneal epithelium stem cell niche. Stem Cells 25: 1145-1155.

Bilic J, Izpisua Belmonte J C (2012), Induced pluripotent stem cells versus embryonic stem cells: close enough or yet too far apart? Stem Cells. 2012 January; 30(1):33-41. doi: 10.1002/stem.700.

Hanson C, Hardarson T, Ellerström C, Nordberg M, Caisander G, Rao M, Hyllner J and Stenevi U (2012), Transplantation of human embryonic stem cells onto a partially wounded human cornea in vitro. Acta Ophthalmol 1-4.

Hayashi R, Ishikawa Y, Ito M, Kageyama T, Takashiba K, et al. (2012) Generation of Corneal Epithelial Cells from Induced Pluripotent Stem Cells Derived from Human Dermal Fibroblast and Corneal Limbal Epithelium. PLoS ONE 7(9): e45435. doi:10.1371/journal.pone.0045435.

Maminishkis A, Chen S, Jalickee S, Banzon T, Shi G, Wang F E, Ehalt T, Hammer J A, and Miller S S (2006), Confluent Monolayers of Cultured Human Fetal Retinal Pigment Epithelium Exhibit Morphology and Physiology of Native Tissue. Invest Ophthalmol Vis Sci. 47(8): 3612-3624.

Osaka F, Ikeda H, Mandai M, Wataya T, Watanabe K, Yoshimura N, Akaike A, Sasai Y, Takahashi M. Toward the Generation of Rod and Cone Photoreceptors from Mouse, Monkey and Human Embryonic Stem Cells. Nat Biotechnol. 2008 February; 26(2):215-24.

Osakada F, Jin Z B, Hirami Y, Ikeda H, Danjyo T, Watanabe K, Sasai Y, Takanashi M. in vitro Differentiation of Retinal cells from Human Pluripotent Stem Cells by Small-Molecule Induction. J Cell Sci. 2009 Sep. 1; 122(pt 17):3169-79.

Rajala, K., Lindroos, B., Hussein, S. Lappalainen, R S., Pekkanen-Mattila, M., Inzunza, H., Miettinen, M., Narkilahti, S., Kerkelä, E., Aalto-Setälä, K., Otonkoski, O., Suuronen, R., Hovatta O., Skottman, H. A Defined and Xeno-free Culture Method Enabling the Establishment of Clinical-grade Human Embryonic, Induced Pluripotent and Adipose Derived Stem Cells. PLoS One. 2010.

Skottman, H. Derivation and characterization of three new human embryonic stem cell lines in Finland. In Vitro Cell Dev Biol Anim. 2010 April; 46(3-4):206-9. Epub 2010 Feb. 23.

Vaajasaari H, Ilmarinen T, Juuti-Uusitalo K, Rajala K, Onnela N, Narkilahti S, Suur-onen R, Hyttinen J, Uusitalo H, Skottman H. Toward the defined and xeno-free differ-entiation of functional human pluripotent stem cell-derived retinal pigment epithelial cells. Mol Vis. 2011 Feb. 22; 17:558-75.

Yoshida S, Yasuda M, Miyashita H, Ogawa Y, Yoshida T, et al. (2011) Generation of Stratified Squamous Epithelial Progenitor Cells from Mouse Induced Pluripotent Stem Cells. PLoS ONE 6(12): e28856. doi:10.1371/journal.pone.0028856.

1.-26. (canceled)

27. A method of producing differentiated eye cells, the method comprising:
a) culturing pluripotent stem cells in an induction medium comprising a TGF-beta inhibitor, a Wnt inhibitor and a fibroblast growth factor for producing eye precursor cells;
b) culturing cells obtained in step a) in a cell culture medium comprising one or more supplements selected from the group consisting of epidermal growth factor (EGF), hydrocortisone, insulin, isoproterenol, and tri-iodo-thyronine, wherein the medium does not contain any of the following supplements: a TGF-beta inhibitor, a Wnt inhibitor and a fibroblast growth factor, for producing differentiated eye cells,
wherein said differentiated eye cells express corneal epithelial marker p63.

28. The method according to claim 27, wherein said differentiated eye cells are selected from the group consisting of corneal epithelial precursor cells and corneal epithelial cells.

29. The method according to claim 27, wherein differentiated eye cells are further maturated into stratified corneal epithelium.

30. The method according to claim 27, wherein said expression of p63 is quantified with immunofluorescent staining

31. The method according to claim 27, wherein the TGF-beta inhibitor is selected from TGF-beta inhibitors having molar mass of less than 800 g/mol.

32. The method according to claim 31, wherein the TGF-beta inhibitor is selected from TGF-beta inhibitors having molar mass of less than 500 g/mol.

33. The method according to claim 27, wherein the TGF-beta inhibitor is selected from organic molecules according to Formula I 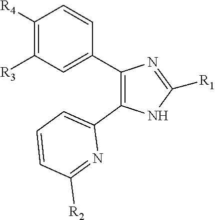
wherein R1 represents an C1-C5 aliphatic alkyl group, carboxylic acid, amide, and R2 represents an C1-C5 aliphatic alkyl, R3 and R4 represent aliphatic alkyls including heteroatoms, O or N, which may be linked together to form a 5- or 6-member heteroring.

34. The method according to claim 27, wherein the Wnt inhibitor is selected from Wnt inhibitors having molar mass of less than 800 g/mol.

35. The method according to claim 34, wherein the Wnt inhibitor is selected from Wnt inhibitors having molar mass of less than 500 g/mol.

36. The method according claim 27, wherein the Wnt inhibitor is selected from organic molecules according to Formula III 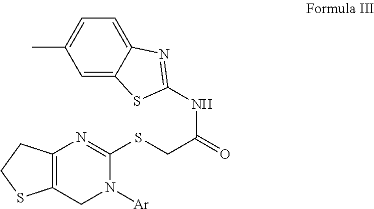
wherein Ar refers to substituted or unsubstituted aryl group.

37. The method according to claim 27, wherein fibroblast growth factor is selected from basic FGF and synthetic small peptides exhibiting fibroblast growth factor-like activity.

38. The method according to claim 27, wherein the content of the TGF-beta inhibitor is from 1 μM to 100 μM, the Wnt-inhibitor is from 1 μM to 100 μM, and the content of fibroblast growth factor is from 1 ng/ml to about 1000 ng/ml.

39. The method according to claim 38, wherein the content of the TGF-beta inhibitor is from 1 to 30 μM, the Wnt-inhibitor is from 1 μM to 30 μM, and the content of fibroblast growth factor is from about 2 ng/ml to about 100 ng/ml.

40. The method according to claim 38, wherein the content of fibroblast growth factor is from about 30 ng/ml to about 80 ng/ml.

41. The method according to claim 27, wherein the stem cells are selected from induced pluripotent stem (iPS) cells and embryonic stem (ES) cells.

42. The method according claim 27, wherein the pluripotent stem cells are cultured for about four to about seven days.

43. The method according to claim 27, wherein said culturing in step b) is carried out on a substrate coated with an extra cellular matrix (ECM) protein selected from collagen IV, collagen I, laminin, vitronectin, fibronectin, and Matrigel™.

44. The method according to claim 27, wherein the corneal epithelial precursor cells expressing said marker p63 represent at least 65%, preferably at least 75%, most preferably at least 90% of the total cell population obtained.

45. The method according to claim 27, which is performed in substantially xeno-free, substantially serum-free and/or defined conditions.

46. A method of producing corneal epithelial precursor cells, corneal epithelial cells, or stratified corneal epithelium, wherein a cell culture medium as defined in step b) of claim 27 is used.
